(19) United States
(12) Patent Application Publication
(10) Pub. No.: US 2025/0302332 A1
(43) Pub. Date: Oct. 2, 2025

(54) MOTION COMPENSATION FOR IMAGING SYSTEM TO SENSOR SYSTEM REGISTRATION AND INSTRUMENT NAVIGATION

(71) Applicant: AURIS HEALTH, INC., Santa Clara, CA (US)

(72) Inventors: Mali Shen, Sunnyvale, CA (US); Namita Anil Kumar, Redwood City, CA (US); Gang Dong, Belmont, CA (US); Hedyeh Rafii-Tari, Mountain View, CA (US); Daniel Shalom Eliahu, Alamo, CA (US)

(73) Assignee: AURIS HEALTH, INC., Santa Clara, CA (US)

(21) Appl. No.: 19/009,494

(22) Filed: Jan. 3, 2025

Related U.S. Application Data
(60) Provisional application No. 63/571,971, filed on Mar. 29, 2024.

(51) Int. Cl. A61B 5/08; A61B 34/20; G06T 7/70

(52) U.S. Cl. CPC A61B 5/0816; A61B 34/20; G06T 7/70

(57) ABSTRACT

This disclosure provides methods, devices, and systems for planning and performing medical procedures. The present implementations more specifically relate to navigating an instrument to a target within an object. In some aspects, a controller for a medical system may capture sensor data, over one or more cyclic movements of an object, via a sensor disposed on the instrument, and may further capture image data via an imaging system external to the object while movement of the object is suspended. The controller determines a baseline pose of the instrument in a sensor space based on the captured sensor data and determines a pose of the instrument in an image space based on the captured image data. The controller further determines a spatial relationship between the instrument and the target based on the baseline pose in the sensor space and the pose of the instrument in the image space.

### CROSS-REFERENCE TO RELATED APPLICATIONS

This application claims priority and benefit under 35 U.S.C. § 119 (c) to U.S. Provisional Patent Application No. 63/571,971, filed Mar. 29, 2024, which is incorporated herein by reference in its entirety.

### TECHNICAL FIELD

This disclosure relates generally to medical systems, and specifically to respiration compensation for imaging system to sensor system registration and instrument navigation.

### DESCRIPTION OF RELATED ART

Many medical procedures include steps that can be performed pre-operation (also referred to as a “preoperative phase”), intra-operation (also referred to as an “intraoperative phase”), or post-operation (also referred to as a “postoperative phase”). For example, during a preoperative phase, an imaging system may be used to scan or otherwise capture images or video of a patient's anatomy. Example suitable imaging technologies include computed tomography (CT), X-ray, fluoroscopy, positron emission tomography (PET), PET-CT, CT angiography, cone beam CT (CBCT), three-dimensional rotational angiography (3DRA), single-photon emission CT (SPECT), magnetic resonance imaging (MRI), optical coherence tomography (OCT), and ultrasound, among other examples. The images may be used, during an intraoperative phase, to help guide or navigate a medical instrument to a target (also referred to as a “treatment site”) within the patient's anatomy. However, images acquired during a preoperative phase may not accurately reflect a spatial relationship between the medical instrument and the target during an intraoperative phase. For example, among various other factors, preoperative images are often acquired several days (or even weeks) before the intraoperative phase, such that changes in the patient's anatomy may cause deviations in the spatial positioning of the target. Thus, there is a need to provide more accurate information about the spatial relationship between the medical instrument and the target during the intraoperative phase.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a method for navigating an instrument within an object (such as an anatomy). The method includes steps of capturing sensor data, over a threshold duration spanning one or more cyclic movements of the object, via a sensor disposed on the instrument; determining a baseline pose of the instrument in a first coordinate space based on the sensor data captured over the threshold duration; capturing image data via an imaging system external to the object while movement is suspended following the threshold duration; determining a pose of the instrument in a second coordinate space based on the image data captured after the threshold duration; and determining a spatial relationship between the instrument and a target within the object based at least in part on the baseline pose of the instrument in the first coordinate space and the pose of the instrument in the second coordinate space. As used herein, the term “cyclic movement” may refer to respiration, heartbeat, or any other physiological movements.

Another innovative aspect of the subject matter of this disclosure can be implemented in a controller for a medical system, including a processing system and a memory. The memory stores instructions that, when executed by the processing system, cause the controller to capture sensor data, over a threshold duration spanning one or more cyclic movements of the object, via a sensor disposed on the instrument; determine a baseline pose of the instrument in a first coordinate space based on the sensor data captured over the threshold duration; capture image data via an imaging system external to the object while movement is suspended following the threshold duration; determine a pose of the instrument in a second coordinate space based on the image data captured after the threshold duration; and determine a spatial relationship between the instrument and a target within the object based at least in part on the baseline pose of the instrument in the first coordinate space and the pose of the instrument in the second coordinate space.

### BRIEF DESCRIPTION OF THE DRAWINGS

The present implementations are illustrated by way of example and are not intended to be limited by the figures of the accompanying drawings.

FIG. 1 shows an example medical system, according to some implementations.

FIG. 2 shows example components of the control system and the robotic system of FIG. 1, according to some implementations.

FIG. 3 shows a block diagram of an example localization system, according to some implementations.

FIG. 4 shows a block diagram of an example navigation controller, according to some implementations.

FIG. 5 shows an example timing diagram depicting changes in pressure inside a patient anatomy during a medical procedure.

FIG. 6A shows example timing diagrams depicting the pose of an instrument in response to changes in pressure.

FIG. 6B shows example frequency diagrams depicting a frequency response of the time-varying poses of the instrument shown in FIG. 6A.

FIG. 7 shows a block diagram of an example controller for a medical system, according to some implementations.

FIG. 8 shows an illustrative flowchart depicting an example operation for navigating an instrument within an object, according to some implementations.

### DETAILED DESCRIPTION

In the following description, numerous specific details are set forth such as examples of specific components, circuits, and processes to provide a thorough understanding of the present disclosure. The term “coupled” as used herein means connected directly to or connected through one or more intervening components or circuits. The terms “electronic system” and “electronic device” may be used interchangeably to refer to any system capable of electronically processing information. Also, in the following description and for purposes of explanation, specific nomenclature is set forth to provide a thorough understanding of the aspects of the disclosure. However, it will be apparent to one skilled in the art that these specific details may not be required to practice the example implementations. In other instances, well-known circuits and devices are shown in block diagram form to avoid obscuring the present disclosure. Some portions of the detailed descriptions which follow are presented in terms of procedures, logic blocks, processing and other symbolic representations of operations on data bits within a computer memory.

These descriptions and representations are the means used by those skilled in the data processing arts to most effectively convey the substance of their work to others skilled in the art. In the present disclosure, a procedure, logic block, process, or the like, is conceived to be a self-consistent sequence of steps or instructions leading to a desired result. The steps are those requiring physical manipulations of physical quantities. Usually, although not necessarily, these quantities take the form of electrical or magnetic signals capable of being stored, transferred, combined, compared, and otherwise manipulated in a computer system. It should be borne in mind, however, that all of these and similar terms are to be associated with the appropriate physical quantities and are merely convenient labels applied to these quantities.

Unless specifically stated otherwise as apparent from the following discussions, it is appreciated that throughout the present application, discussions utilizing the terms such as “accessing,” “receiving,” “sending,” “using,” “selecting,” “determining,” “normalizing,” “multiplying,” “averaging,” “monitoring,” “comparing,” “applying,” “updating,” “measuring,” “deriving” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

Certain standard anatomical terms of location may be used herein to refer to the anatomy of animals, and namely humans, with respect to the example implementations. Although certain spatially relative terms, such as “outer,” “inner,” “upper,” “lower,” “below,” “above,” “vertical,” “horizontal,” “top,” “bottom,” and similar terms, are used herein to describe a spatial relationship of one element, device, or anatomical structure to another device, element, or anatomical structure, it is understood that these terms are used herein for ease of description to describe the positional relationship between elements and structures, as illustrated in the drawings. It should be understood that spatially relative terms are intended to encompass different orientations of the elements or structures, in use or operation, in addition to the orientations depicted in the drawings. For example, an element or structure described as “above” another element or structure may represent a position that is below or beside such other element or structure with respect to alternate orientations of the subject patient, element, or structure, and vice-versa. As used herein, the term “patient” may generally refer to humans, anatomical models, simulators, cadavers, and other living or non-living objects.

In the figures, a single block may be described as performing a function or functions; however, in actual practice, the function or functions performed by that block may be performed in a single component or across multiple components, or may be performed using hardware, using software, or using a combination of hardware and software. To clearly illustrate this interchangeability of hardware and software, various illustrative components, blocks, modules, circuits, and steps have been described below generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure. Also, the example systems or devices may include components other than those shown, including well-known components such as a processor, memory and the like.

The techniques described herein may be implemented in hardware, software, firmware, or any combination thereof, unless specifically described as being implemented in a specific manner. Any features described as modules or components may also be implemented together in an integrated logic device or separately as discrete but interoperable logic devices. If implemented in software, the techniques may be realized at least in part by a non-transitory processor-readable storage medium including instructions that, when executed, performs one or more of the methods described herein. The non-transitory processor-readable data storage medium may form part of a computer program product, which may include packaging materials.

The non-transitory processor-readable storage medium may comprise random access memory (RAM) such as synchronous dynamic random-access memory (SDRAM), read only memory (ROM), non-volatile random access memory (NVRAM), electrically erasable programmable read-only memory (EEPROM), FLASH memory, other known storage media, and the like. The techniques additionally, or alternatively, may be realized at least in part by a processor-readable communication medium that carries or communicates code in the form of instructions or data structures and that can be accessed, read, or executed by a computer or other processor.

The various illustrative logical blocks, modules, circuits and instructions described in connection with the implementations disclosed herein may be executed by one or more processors (or a processing system). The term “processor,” as used herein may refer to any general-purpose processor, special-purpose processor, conventional processor, controller, microcontroller, or state machine capable of executing scripts or instructions of one or more software programs stored in memory.

As described above, many medical procedures include a preoperative phase that precedes an intraoperative phase. During the preoperative phase, for some medical procedures, an imaging system may be used to scan or otherwise capture images or video of at least a portion of a patient's anatomy. For example, a computed tomography (CT) scanner may be used to acquire tomographic images (also referred to as “tomograms” or “CT scans”) of a patient's lungs during the preoperative phase for a bronchoscopy. A tomogram is a cross-section or slice of a three-dimensional (3D) volume. For example, multiple tomograms can be stacked or combined to recreate the 3D volume (such as a 3D model of the patient's lungs). Thus, tomograms can be used to detect a precise location or position (in 3D space) of a nodule or target in the patient's lungs. During the intraoperative phase, for some medical procedures, a medical system may use the preoperative images to generate a graphical interface for navigating a medical instrument within the patient's anatomy. For example, during a bronchoscopy, the medical system may detect a pose of an endoscope (such as a position and orientation of the scope in 3D space) based on sensor data received via an electromagnetic (EM) sensor disposed on the tip of the scope and map the pose of the endoscope to a 3D model of the patient's lungs depicted by the tomograms.

Accordingly, the graphical interface may depict a spatial relationship between the medical instrument and the target within the anatomy based on the sensor data and the image data. However, images acquired during a preoperative phase may not accurately reflect the spatial relationship between the medical instrument and the target during an intraoperative phase. For example, changes in the patient's anatomy or the medical environment can cause the spatial relationship between the endoscope and target to deviate from what is depicted by the graphical interface at any given time, which can lead to inaccurate navigation. Example factors include EM distortion, poor registration (or mapping) between the sensor space and the image space associated with the preoperative scans (also referred to as the “preoperative image space”), outdated preoperative scans, and anatomical deformations, among other examples. Aspects of the present disclosure recognize that some modern imaging technologies (such as cone beam CT) can be used to scan a patient's anatomy during an intraoperative phase. In some aspects, a medical system may capture updated images of the patient's anatomy during the intraoperative phase and use the updated image data to improve the representation of the spatial relationship between the medical instrument and the target.

The updated image data and sensor data are often associated with different coordinate spaces. Thus, in some implementations, the medical system may “register” the updated image space with the sensor space to facilitate real-time navigation. As used herein, the term “registration” refers to a mapping or transformation between different coordinate spaces. For example, a medical system may register an imaging system used for capturing images of a patient's anatomy (such as a cone beam CT scanner) with a sensor system used for tracking a pose of a medical instrument within the anatomy (such as an EM field generator) by determining a mapping or spatial transformation that maps any point or vector in the image space to a respective point or vector in the sensor space (such as a transformation matrix). The terms “mapping,” “transformation,” “spatial transformation,” and “registration matrix,” may be used interchangeably herein. The terms “respective” and “corresponding” also may be used interchangeably herein.

In some medical procedures (such as bronchoscopy), the patient is under general anesthesia and allowed to breathe through a ventilator. However, because an imaging system may capture multiple images (such as tomograms) when scanning the patient's anatomy, any changes to the anatomy during the course of the scan (such as due to respiration) may introduce artifacts or other inaccuracies in the resulting images. To acquire accurate scans of a patient's anatomy during an intraoperative phase of a medical procedure, the patient may be prevented from breathing for the duration of the scan (such as by forcing a breath-hold via the ventilator). Aspects of the present disclosure recognize that respiration can change the pose (including the position and/or orientation) of a medical instrument inside the patient's anatomy. As a result, the pose of an instrument while respiration is suspended may differ from the pose of the instrument, at any given time, while the patient is breathing (even when no user inputs are applied to the instrument). Aspects of the present disclosure further recognize that some imaging technologies (such as CT or X-rays) may interfere with some sensor technologies (such as EM). Thus, sensor data acquired before scanning an anatomy may deviate from sensor data acquired after the scan. In some aspects, a medical system may compensate or otherwise account for any deviations in instrument pose caused by sensor interference or changes in anatomy when registering an image space with a sensor space and applying the registration to real-time sensor data.

Although certain aspects of the present disclosure are described in detail herein in the context of bronchoscopy, it should be understood that the systems and techniques of the present disclosure may be applicable to any medical procedure. Example medical procedures may include minimally invasive procedures (such as laparoscopy), non-invasive procedures (such as endoscopy), therapeutic procedures, diagnostic procedures, percutaneous procedures, and non-percutaneous procedures, among other examples. Example endoscopic procedures include bronchoscopy, ureteroscopy, gastroscopy, nephroscopy, and nephrolithotomy, among other examples. The terms “scope,” “endoscope,” “catheter,” and “instrument” may be used interchangeably herein.

Aspects of the present disclosure may be used to perform robotic-assisted medical procedures, such as endoscopic access, percutaneous access, or treatment for a target anatomical site. For example, robotic tools may engage or control one or more medical instruments (such as an endoscope) to access a target site within a patient's anatomy or perform a treatment at the target site. In some implementations, the robotic tools may be guided or controlled by a physician. In some other implementations, the robotic tools may operate in an autonomous or semi-autonomous manner. Although systems and techniques are described herein in the context of robotic-assisted medical procedures, the systems and techniques may be applicable to other types of medical procedures (such as procedures that do not rely on robotic tools or only utilize robotic tools in a very limited capacity). For example, the systems and techniques described herein may be applicable to medical procedures that rely on manually operated medical instruments (such as an endoscope that is exclusively controlled and operated by a physician). The systems and techniques described herein also may be applicable beyond the context of medical procedures (such as in simulated environments or laboratory settings, such as with models or simulators, among other examples).

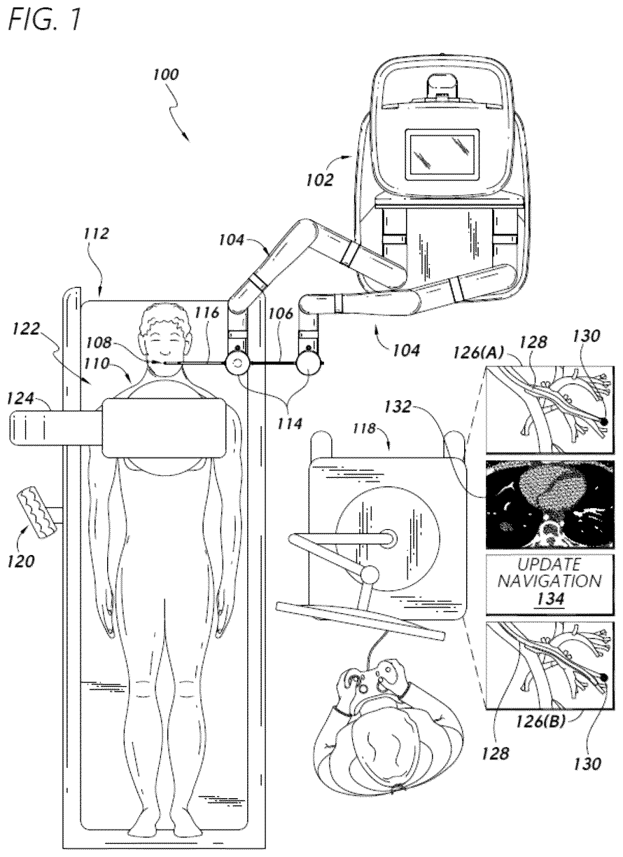

FIG. 1 shows an example medical system 100 (also referred to as a “surgical medical system” or a “robotic medical system”), according to some implementations. As shown in FIG. 1, the medical system 100 may be arranged for diagnostic or therapeutic bronchoscopy. The medical system 100 can include and utilize a robotic system 102 which can be implemented, for example, as a robotic cart. Although the medical system 100 is shown as including various cart-based systems or devices, the concepts disclosed herein can be implemented in any type of robotic system or arrangement, such as robotic systems employing rail-based components, table-based robotic end-effectors, or manipulators, among other examples. The robotic system 102 may include one or more robotic arms 104 (also referred to as “robotic positioners”) configured to position or otherwise manipulate a medical instrument 106 (such as a steerable endoscope or another elongate instrument). For example, the medical instrument 106 can be advanced through a natural orifice access point (such as the mouth 108 of a patient 110 positioned on a table 112) to deliver diagnostic or therapeutic treatment. Although described in the context of a bronchoscopy procedure, the medical system 100 also may be used to perform other types of medical procedures. Example suitable procedures include gastro-intestinal (GI) procedures, renal procedures, urological procedures, and nephrological procedures, among other examples.

With the robotic system 102 properly positioned, the medical instrument 106 can be inserted into the patient 110 robotically, manually, or a combination thereof. For example, the one or more robotic arms 104, or instrument drivers 114 coupled thereto, can control the medical instrument 106. In some implementations, the medical instrument 106 may be advanced within a sheath 116. For example, the sheath 116 may be coupled to, or controlled by, a robotic arm 104. In some implementations, the medical instrument 106 and the sheath 116 may each be coupled to a respective instrument driver from a set of instrument drivers 114. The instrument drivers 114 can be repositionable in space by manipulating the one or more robotic arms 104 into different angles or positions.

In the example of FIG. 1, the medical instrument 106 can be directed down the patient's trachea and lungs after insertion or advanced to a target destination or operative site. In some implementations, to enhance navigation through the patient's lung network or reach the desired target, the medical instrument 106 may be manipulated to telescopically extend from the outer sheath 116 to obtain enhanced articulation or greater bend radius. The use of separate instrument drivers 114 can allow the medical instrument 106 and sheath 116 to be driven independently of each other.

In some implementations, the medical instrument 106 may include an elongate member or shaft configured to be inserted or retracted, articulated, or otherwise moved within the anatomy. Further, in some implementations, the medical instrument 106 may include one or more imaging devices (such as cameras) positioned on a distal end of the elongate shaft or deployed through a working channel of the elongate shaft. The imaging devices can be configured to generate or capture image (or video) data or send the image data to another device or component. In some implementations, the medical instrument 106 may include an instrument base or one or more handles positioned at a proximal end of the medical instrument 106. The instrument base can be coupled to a manipulator (such as an end of a robotic arm 104). The instrument base can include one or more drive inputs coupled to one or more drive outputs of the manipulator, wherein the drive inputs or drive outputs act as an interface.

In some implementations, the medical instrument 106 may include a working channel configured to receive one or more other instruments or elements therein or provide other functionality. The working channel can extend axially, such as along the length of the medical instrument 106. Furthermore, the medical instrument 106 can include or be associated with one or more elongate movement members (such as pulls wires) that can extend from a proximal end through the elongate shaft to the distal end of the elongate shaft. The elongate movement members can be manipulated, such as by manipulators on the one or more robotic arms 104, to control actuation of the elongate movement members.

In some implementations, the medical instrument 106 may include one or more sensors, such as electromagnetic (EM) sensors, shape sensors (such as shape sensing fiber), accelerometers, gyroscopes, satellite-based positioning sensors (such as global positioning system (GPS) sensors), or radio-frequency (RF) transceivers, among other examples. The sensors can be configured to generate or produce sensor data or provide the sensor data to another device or component. The sensors can be disposed at a distal end of the elongate shaft or along a length of the elongate shaft. In some implementations, the medical instrument 106 may be configured to receive an elongate member or device through a working channel, wherein the elongate member includes one or more sensors along a length of the elongate member. One or more sensors on the medical instrument 106 may provide sensor data to control circuitry of the medical system 100, which is then used to determine a position, orientation, or shape of the medical instrument 106.

The medical system 100 can also include a control system 118 (also referred to as a “control tower” or “mobile tower”). The control system 118 can be communicatively coupled (such as via wired or wireless connections) to the robotic system 102 to control various aspects of the robotic system 102 (such as electronics, optics, sensors, or power) or one or more subsystems associated with the robotic system 102, such as a fluid management system (not shown). Placing such functionality in the control system 118 can allow for a smaller form factor of the robotic system 102 that may be more easily adjusted or re-positioned by an operator or user. Additionally, the division of functionality between the robotic system 102 and the control system 118 can reduce operating room clutter and facilitate efficient clinical workflow.

The medical system 100 can include an electromagnetic (EM) field generator 120, which is configured to broadcast or emit an EM field that can be detected by various EM sensors, such as a sensor disposed on the medical instrument 106. The EM field can induce small electric currents in coils of the EM sensors, which can be analyzed to determine a position, angle, or orientation of the EM sensors relative to the EM field generator 120. Although EM fields and EM sensors are described in many examples herein, position sensing systems or sensors can include various other types of position sensing systems or sensors, such as optical position sensing systems or sensors, image-based position sensing systems or sensors, among other examples.

The medical system 100 can further include an imaging system 122 (also referred to as an “imaging device”) configured to generate, provide, or send image data (also referred to as “images”) to another device or system. For example, the imaging system 122 can generate image data depicting an anatomy of the patient 110 and provide the image data to the control system 118, the robotic system 102, or another device. The imaging system 122 may include an emitter or energy source (such as an X-ray source) or a detector (such as an X-ray detector) mounted on a C-shaped arm support 124, which allows for flexibility in positioning around the patient 110 to capture images from various angles without moving the patient 110. Use of the imaging system 122 can provide visualization of internal structures or anatomy, which can be used for a variety of purposes, including navigation of the medical instrument 106 (such as by providing images of internal anatomy to a user) and localization of the medical instrument 106 (based on an analysis of image data), among other examples. In some aspects, the imaging system 122 may enhance the efficacy or safety of a medical procedure, such as a bronchoscopy, by providing clear, continuous visual feedback to the operating surgeon or team.

In some implementations, the imaging system 122 may be a mobile device configured to move around an environment. For example, the imaging system 122 can be positioned next to the patient 110 (as shown in FIG. 1) during a particular phase of a procedure and removed when the imaging system 122 is no longer needed. In some other implementations, the imaging system 122 may be part of the table 112 or other equipment in an operating environment. The imaging system 122 can be implemented as a Computed Tomography (CT) machine or system, X-ray machine or system, fluoroscopy machine or system, Positron Emission Tomography (PET) machine or system, PET-CT machine or system, CT angiography machine or system, Cone-Beam CT (CBCT) machine or system, three-dimensional rotational angiography (3DRA) machine or system, single-photon emission computed tomography (SPECT) machine or system, Magnetic Resonance Imaging (MRI) machine or system, Optical Coherence Tomography (OCT) machine or system, or ultrasound machine or system, among other examples. In some implementations, the medical system 100 may include different types of imaging systems that can be used or positioned over the patient 110 during different phases or portions of a procedure depending on the needs at that time.

In some implementations, the imaging system 122 may be configured to process multiple images (also referred to as “image data”) to generate a three-dimensional (3D) view or model. For example, the imaging device 122 can be implemented as a CT machine configured to capture or generate a series of images (also referred to as “tomograms) or image data representing two-dimensional (2D) cross-sections or slices of a 3D volume from different angles around the patient 110, and then use one or more algorithms to reconstruct these images or image data into a 3D model. The 3D model can be provided to the control system 118, robotic system 102, or another device, such as for processing or display.

In some implementations, image data from the imaging system 122 may be used to localize various elements, such as the medical instrument 106, a target within the anatomy, or specific anatomical features, among other examples. For example, the control system 118 can be configured to provide navigation information during a procedure to assist a user navigating the medical instrument 106 within the anatomy to reach a target (such as a desired treatment site or location). In some implementations, a target can include a nodule, such as in the context of certain bronchoscopy procedures. To illustrate, the control system 118 can display a navigation view or graphical data 126 that includes an instrument indicator 128 representing the medical instrument 106, a target indicator 130 representing the target, and an anatomical map. The navigation data 126(A) (such as initial navigation data) can be determined based on sensor data from a sensor of the medical instrument 106 (such as EM sensor data associated with the EM field generator 120), a map of the anatomy, or a location of the target. In some implementations, the map or location of the target may be determined based on preoperative data, such as data obtained during a preoperative procedure to find a target location or map the anatomy.

In some implementations, the navigation data 126(A) may be dynamically updated based on image data 132 from the imaging system 122. For example, the control system 118 can receive the image data 132 and analyze the image data 132 to determine a current or actual spatial relationship between the medical instrument 106 and the target. In some implementations, the control system 118 may display the image data 132 to a user, receive user input indicating a position of the medical instrument 106 or a position of the target in the image data 132, and analyze the image data 132 based on the user input to determine the current spatial relationship. If the control system 118 determines that the navigation data 126(A) incorrectly depicts the location of the medical instrument 106 (such as where the spatial relationship associated with the image data 132 is different than the spatial relationship associated with the navigation data 126(A)), the control system 118 may update the navigation data 126(A) at 134 and provide updated navigation data 126(B) that reflects the current or near real-time position of the medical instrument 106 relative to the target or the map.

The various components of the medical system 100 can be communicatively coupled to each other over a network, which can include a wireless or wired network. Example networks include one or more personal area networks (PANs), local area networks (LANs), wide area networks (WANs), Internet area networks (IANs), cellular networks, the Internet, personal area networks (PANs), body area network (BANs), etc. In some examples, various communication interfaces can include wireless technology, such as Bluetooth, Wi-Fi, near-field communication (NFC), or the like. Furthermore, in some examples, the various components of the medical system 100 can be connected for data communication, fluid exchange, power exchange, and so on, via one or more support cables, tubes, connections, or the like.

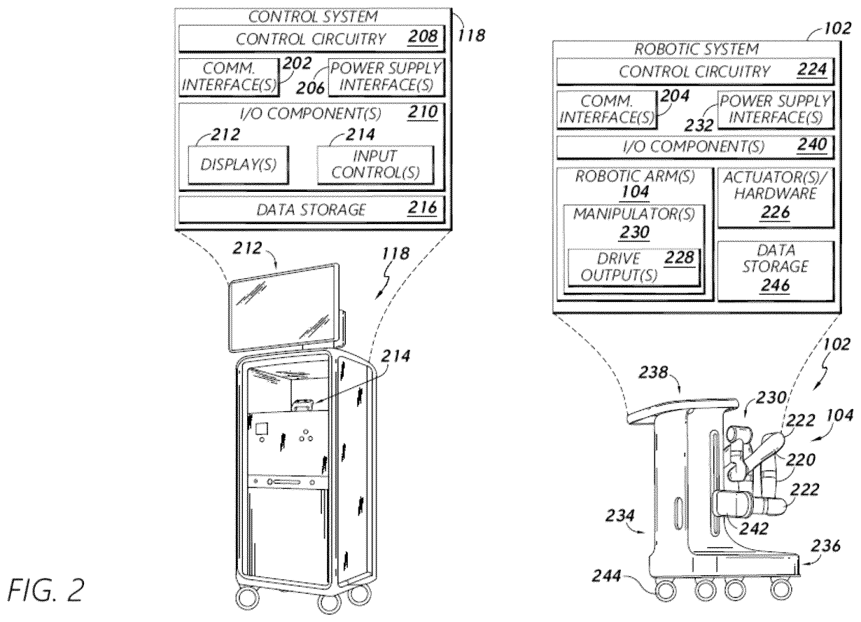

FIG. 2 shows example components of the control system 118 and the robotic system 102 of FIG. 1, according to some implementations. In the examples of FIG. 2, the control system 118 and the robotic system 102 are implemented as a tower and a robotic cart, respectively. However, the control system 118 and robotic system 102 can be implemented in other manners. The control system 118 can be coupled to the robotic system 102 and operate in cooperation therewith to perform a medical procedure. For example, the control system 118 can include communication interface(s) 202 for communicating with communication interface(s) 204 of the robotic system 102 via a wireless or wired connection (such as to control the robotic system 102). In some implementations, the control system 118 may communicate with the robotic system 102 to receive position or sensor data therefrom relating to the position of sensors associated with an instrument or member controlled by the robotic system 102. For example, the control system 118 may communicate with the EM field generator 120 to control generation of an EM field in an area around a patient. The control system 118 can further include one or more power supply interface(s) 206.

The control system 118 can include control circuitry 208 configured to cause one or more components of the medical system 100 to actuate or otherwise control any of the various system components, such as carriages, mounts, arms or positioners, medical instruments, imaging devices, position sensing devices, or sensors, among other examples. Further, the control circuitry 208 can be configured to perform other functions, such as cause display of information, process data, receive input, communicate with other components or devices, or any other function or operation described herein.

The control system 118 can further include one or more input or out (I/O) components 210 configured to assist a physician or others in performing a medical procedure. For example, the one or more I/O components 210 can be configured to receive input or provide output to enable a user to control or navigate the medical instrument 106, the robotic system 102, or other instruments or devices associated with the medical system 100. The control system 118 can include one or more displays 212 to provide, display or otherwise present various information regarding a procedure. For example, the one or more displays 212 can be used to present navigation information including a virtual anatomical model of anatomy with a virtual representation of a medical instrument, image data, or other information. The one or more I/O components 210 can include one or more user input control(s) 214, which can include any type of user input (or output) devices or device interfaces, such as one or more buttons, keys, joysticks, handheld controllers (such as video-game-type controllers), computer mice, trackpads, trackballs, control pads, sensors (such as motion sensors or cameras) that capture hand gestures and finger gestures, touchscreens, toggle (such as button) inputs, or interfaces or connectors therefore. In some implementations, such inputs can be used to generate commands for controlling one or more medical instruments, robotic arms, or other components.

The control system 118 can also include data storage 216 configured to store executable instruments (such as computer-readable instructions) that can be executed by the control circuitry 208 to cause the control circuitry 208 to perform various operations or functionality described herein. In some implementations, the data storage 216 also may store telemetry or runtime data (such as sensor data or image data) generated by the medical system 100 or otherwise captured or acquired during a medical procedure. In some implementations, two or more components of the control system 118 can be electrically or communicatively coupled to each other.

The robotic system 102 can include the one or more robotic arms 104 configured to engage with or control, for example, the medical instrument 106 or other elements or components to perform one or more aspects of a procedure. As shown in FIG. 2, each robotic arm 104 can include multiple segments 220 coupled to joints 222, which can provide multiple degrees of movement or freedom. The robotic system 102 can be configured to receive control signals from the control system 118 to perform certain operations, such as to position one or more of the robotic arms 104 in a particular manner or manipulate an instrument, among other examples. In response, the robotic system 102 can control, using control circuitry 224 thereof, actuators 226 or other components of the robotic system 102 to perform the operations. For example, the control circuitry 224 can control insertion or retraction, articulation, or roll of a shaft of the medical instrument 106 or other instrument by actuating one or more drive outputs 228 of a manipulator 230 (or end-effector) coupled to a base of a robotically-controllable instrument. The drive outputs 228 can be coupled to a drive input on an associated instrument, such as an instrument base of an instrument that is coupled to the associated robotic arm 104. The robotic system 102 also may include one or more power supply interfaces 232.

The robotic system 102 can include a support column 234, a base 236, or a console 238. The console 238 can provide one or more I/O components 240, such as a user interface for receiving user input or a display screen (or a dual-purpose device, such as a touchscreen) to provide the physician or user with preoperative or intraoperative data. The support column 234 can include an arm support 242 (also referred to as a “carriage”) for supporting the deployment of the one or more robotic arms 104. The arm support 242 can be configured to vertically translate along the support column 234. Vertical translation of the arm support 242 allows the robotic system 102 to adjust the reach of the robotic arms 104 to meet a variety of table heights, patient sizes, or physician preferences. The base 236 can include wheel-shaped casters 244 (also referred to as “wheels”) that allow the robotic system 102 to move around the operating room. After reaching the appropriate position, the casters 244 can be immobilized using wheel locks to hold the robotic system 102 in place during the procedure.

The joints 222 of each robotic arm 104 can each be independently-controllable or provide an independent degree of freedom available for instrument navigation. In some implementations, each robotic arm 104 may include seven joints that provide seven degrees of freedom, including “redundant” degrees of freedom. Redundant degrees of freedom can allow robotic arms 104 to be controlled to position their respective manipulators 230 at a specific position, orientation, or trajectory in space using different linkage positions and joint angles. This allows for the robotic system 102 to position or direct a medical instrument from a desired point in space while allowing the physician to move the joints 222 into a clinically advantageous position away from the patient to create greater access, while avoiding collisions.

The one or more manipulators 230 (or end-effectors) can be coupled to an instrument base or handle, which can be attached using a sterile adapter component. The combination of the manipulator 230 and instrument base, as well as any intervening mechanics or couplings (such as the sterile adapter), can be collectively referred to as the manipulator or a manipulator assembly. Manipulators or manipulator assemblies can provide power or control interfaces. Example interfaces may include connectors to transfer pneumatic pressure, electrical power, electrical signals, or optical signals from the robotic arm 104 to an instrument base. Manipulators or manipulator assemblies can be configured to manipulate medical instruments (such as surgical tools) using techniques including, for example, direct drives, harmonic drives, geared drives, belts or pulleys, or magnetic drives, among other examples.

The robotic system 102 can also include data storage 246 configured to store executable instruments (such as computer-readable instructions) that can be executed by the control circuitry 224 to cause the control circuitry 224 to perform various operations or functionality described herein. In some implementations, the data storage 216 also may store telemetry or runtime data (such as sensor data or image data) generated by the medical system 100 or otherwise captured or acquired during a medical procedure. In some implementations, two or more of the components of the robotic system 102 can be electrically or communicatively coupled to each other.

Data storage (including the data storage 216, data storage 246, or other data storage or memory) can include any suitable or desirable type of computer-readable media. For example, computer-readable media can include one or more volatile data storage devices, non-volatile data storage devices, removable data storage devices, or nonremovable data storage devices implemented using any technology, layout, or data structure(s) or protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Functionality described herein can be implemented by the control circuitry 208 of the control system 118 or the control circuitry 224 of the robotic system 102, such as by the control circuitry 208 or 224 executing instructions to cause the control circuitry 208 or 224 to perform the functionality. Control circuitry (including the control circuitry 208, control circuitry 224, or other control circuitry) can include circuitry embodied in a robotic system, control system or tower, instrument, or any other component or device. Control circuitry can include any collection of processors, processing circuitry, processing modules or units, chips, dies (such as semiconductor dies including one or more active or passive devices or connectivity circuitry), microprocessors, micro-controllers, digital signal processors, microcomputers, central processing units, field-programmable gate arrays, programmable logic devices, state machines (such as hardware state machines), logic circuitry, analog circuitry, digital circuitry, or any device that manipulates signals (analog or digital) based on hard coding of the circuitry or operational instructions.

Control circuitry referenced herein can further include one or more circuit substrates (such as printed circuit boards), conductive traces and vias, or mounting pads, connectors, or components. Control circuitry can further include one or more storage devices, which may be embodied in a single device, a plurality of devices, or embedded circuitry of a device. Such data storage can comprise read-only memory, random access memory, volatile memory, non-volatile memory, static memory, dynamic memory, flash memory, cache memory, data storage registers, or any device that stores digital information. In examples in which control circuitry includes a hardware or software state machine, analog circuitry, digital circuitry, or logic circuitry, data storage device(s) or register(s) storing any associated operational instructions can be embedded within, or external to, the circuitry comprising the state machine, analog circuitry, digital circuitry, or logic circuitry.

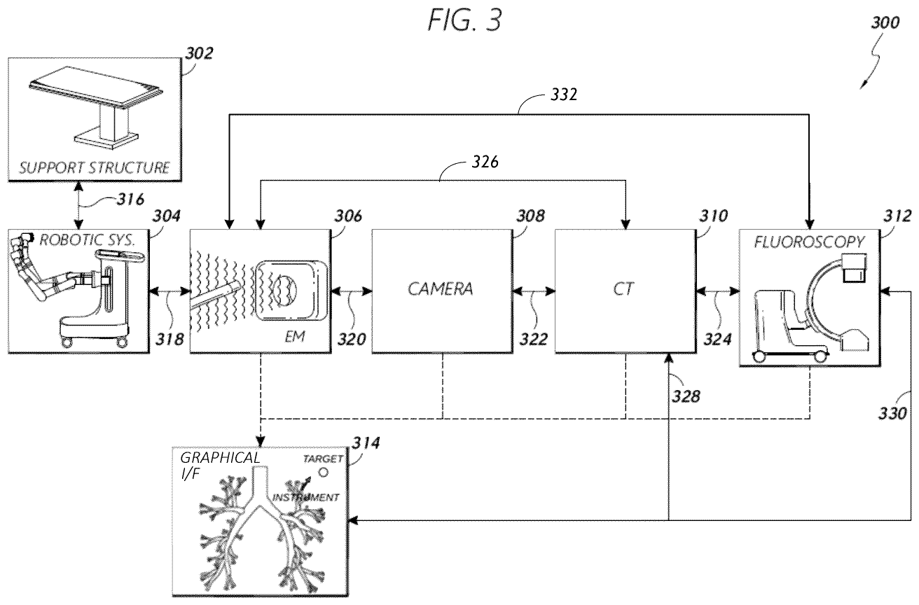

FIG. 3 shows a block diagram of an example localization system 300, according to some implementations. The localization system 300 includes various positioning or imaging systems or modalities 302-312 (also referred to as “subsystems”), which can be implemented to facilitate anatomical mapping, navigation, positioning, or visualization for procedures in accordance with one or more examples. For example, the various systems 302-312 can be configured to provide data for generating an anatomical map, determining a location of an instrument, determining a location of a target, or performing other techniques.

Each of the systems 302-312 can be associated with a respective coordinate space (also referred to as a “position coordinate frame”) or can provide data or information relating to instrument or anatomy locations, wherein registering the various coordinate spaces to one another can allow for integration of the various systems to provide mapping, navigation, or instrument visualization. For example, registering a first modality to a second modality can allow for determined positions in the first modality to be tracked or superimposed on or in a reference frame associated with the second modality, thereby providing layers of positional information that can be combined to provide a robust localization system.

In some implementations, the system 300 may be configured to implement one or more localization or localizing techniques. As used herein, the terms “localization” or “localizing” refer to any processes for determining an instrument position (or location) and orientation (or heading), collectively referred to as the “pose” of the instrument or other element or component, within a given space or environment.

In some implementations, the anatomical space in which a medical instrument can be localized (such as where a position or shape of the instrument is determined or estimated) may be a 2D or 3D portion of a patient's tracheobronchial airways, vasculature, urinary tract, gastrointestinal tract, or any organ or space accessed via lumens. Various modalities can be implemented to provide images, representations, or models of the anatomical space. For example, an imaging modality can be implemented, which can include, for example, X-ray, fluoroscopy, CT, PET, PET-CT, CT angiography, CBCT, 3DRA, SPECT, MRI, OCT, or ultrasound, among other examples. In some implementations, the imaging modality may be used to capture or acquire images of a patient's anatomy during a preoperative phase of a medical procedure. In some other implementations, the imaging modality may be used to capture or acquire images of a patient's anatomy during an intraoperative phase of the medical procedure.

The systems 302-312 can provide information for generating a graphical user interface 314 (also referred to as a “graphical interface (I/F)”) that includes navigation information for navigating an instrument to a target within an anatomy (such as the navigation data 126(A) or 126(B) of FIG. 1). For example, the navigation information may include an anatomical map, an estimated position, orientation, or shape of the instrument, and/or a position of the target. In some implementations, the graphical user interface 314 or other localization information may be displayed to a user, such as a physician, during a medical procedure to assist the user in performing the procedure. For example, a visualization of a tracked instrument can be superimposed on an anatomical map depicted by the graphical user interface 314 based on position or sensor data associated with the tracked medical instrument.

As shown in FIG. 3, the system 300 can include a support structure 302 (such as a surgical bed or other patient positioning or support platform). For example, the support structure 302 includes a planar surface that contacts and supports the patient. In some implementations, the position of the support structure 302 may be known based on data maintained relating to the position of the support structure 302 within the surgical or procedure environment. In some other implementations, the position of the support structure 302 may be sensed or otherwise determined using one or more markers or an appropriate imaging or positioning modality.

The system 300 can further include a robotic system 304 (such as a robotic cart or other device or system including one or more robotic end effectors). In some implementations, the robotic system 304 may be one example of the robotic system 102 of FIGS. 1 and 2. Data relating to the position or state of robotic arms, actuators, or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration, in some implementations, based on robotic system or cart-set-up procedure (which can have any suitable or desirable scheme).

The system 300 can further include an electromagnetic (EM) sensor system 306, which can include an EM field generator (such as the EM field generator 120 of FIG. 1) and one or more EM sensors. An EM sensor can be associated with a portion of an instrument that is tracked or controlled, such as a distal end (or tip) of the instrument or along a length of the instrument or other elongate member (such as a working channel) disposed in a lumen of the instrument. In some implementations, the EM field generator can be mechanically coupled to the support structure 302 or the robotic system 304 such that registration or association 318 between such systems can be known or determined. In some implementations, the registration 318 between the EM sensor system 306 and the robotic system 304 can be determined through forward kinematics or field generator mount transform information. For example, the field generator can be mounted to the support structure 302 such that the position of the field generator can be known relative to the robotic system positioning frame based on a known relationship between the position of the support structure 302 and the robotic system 304. The EM sensor system 306 can provide instrument pose or path information based on sensor readings associated with the instrument.

The system 300 can further include an optical camera system 308 including one or more cameras or other imaging devices configured to generate images of patient anatomy within a visual field thereof (such as real-time image data) during a surgical procedure. In some implementations, registration 320 between the optical camera system 308 and the EM sensor system 306 can be achieved through identification of features having EM sensor data associated therewith, such as a medical instrument tip, in images generated by the optical camera system 308. The registration 320 can further be based at least in part on hand-eye interaction of the physician when viewing real-time camera images while the EM-sensor-equipped endoscope is navigating in the patient anatomy.

The system 300 can further include a computed tomography (CT) imaging system 310 configured to generate CT images of the patient anatomy, which can be performed preoperatively or intraoperatively. The CT imaging system 310 is generally used for scanning a relatively large volume. In some implementations, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the graphical user interface 314 or for path navigation planning.

In some aspects, the CT imaging system 310 may be registered 326 to the EM sensor system 306 through various techniques, such as tool registration or a transformation function, among other examples. In some implementations, a mechanical structure of the CT imaging system 310 can have a known physical transform or relationship with respect to a mounting position of the EM field generator of the EM sensor system 306. Such known relationship can be used to register the CT image space to the EM sensor space. The connection 328 represents a mapping or relationship between the CT imaging system 310 and an anatomical map depicted by a graphical user interface 314.

The system 300 can further include a fluoroscopy imaging system 312 configured to generate tomographic images (such as real-time X-ray images) of the surgical site. The fluoroscopy imaging system 312 is generally used for scanning a smaller volume compared to the CT imaging system 310. In some implementations, the fluoroscopy imaging system 312 may be one example of the imaging system 122 of FIG. 1. For example, the fluoroscopy imaging system 312 may include a CBCT scanner coupled to a C-arm. In some implementations, the fluoroscopy imaging system 312 may be used with a contrast agent introduced into the anatomy to generate image data representing patient anatomy or instrumentation. In some implementations, the fluoroscopy imaging system 312 may be registered 324 to the CT imaging system 310 using any image processing technique suitable for such registration.

In some aspects, the fluoroscopy imaging system 312 may be registered 332 to the EM sensor system 306 through various techniques, such as tool registration or a transformation function, among other examples. In some implementations, a mechanical structure of the fluoroscopy imaging system 312 (such as the C-arm instrumentation) can have a known physical transform or relationship with respect to a mounting position of the EM field generator of the EM sensor system 306. Such known relationship can be used to register the fluoroscopy image space to the EM sensor space. The connection 330 represents a mapping or relationship between the fluoroscopy imaging system 312 and an anatomical map depicted by the graphical user interface 314.

In the example of FIG. 3, the CT imaging system 310 and fluoroscopy imaging system 312 are illustrated as separated systems. However, in some other implementations, a single imaging system may perform the functions of both the CT imaging system 310 and fluoroscopy imaging system 312.

The position, shape, or orientation of an instrument, such as an endoscope, can be determined using any one or more of the systems 302-312, which can facilitate generation of graphical interface data representing the estimated position or shape of the instrument relative to an anatomical map depicted by the graphical user interface 314. The graphical user interface 314 can be displayed on a display device, such as via the control system 118 or robotic system 102, or another device. In some implementations, the graphical user interface 314 also may indicate a position of a target within the anatomy that has been designated for treatment.

Although the systems 302-312 have been described in a particular order, the operations or functions associated therewith can be performed in different orders. In some implementations, the systems 302-312 can be used in different ways. In some other implementations, registration can occur between different systems and modalities.

In some aspects, one or more of the systems 302-312 may be used to generate the graphical user interface 314 preoperatively or determine a location of one or more targets within an anatomical map depicted by the graphical user interface 314 during a preoperative phase of a medical procedure. However, a graphical user interface 314 generated using a preoperative CT scan may not accurately reflect the spatial relationship between a medical instrument and a target during an intraoperative phase. For example, changes in the patient's anatomy or the medical environment can cause the spatial relationship between the instrument and the target to deviate from what is depicted in the graphical user interface 314. Example factors that may cause such deviations include EM distortion, poor registration (or mapping) between the sensor data and the image data, outdated preoperative scans, and anatomical deformations, among other examples. Thus, in some other aspects, one or more of the systems 302-312 may be used to determine a location of a medical instrument or position of a target relative to an anatomical map depicted by the graphical user interface 314 during an intraoperative phase of the medical procedure.

As described with reference to FIG. 3, image data and sensor data are often associated with different coordinate spaces. For example, the image data may describe data points (such as coordinates or vectors) in relation to a coordinate space defined by an imaging system (such as the fluoroscopy imaging system 312) whereas the sensor data may describe data points (such as coordinates or vectors) in relation to a coordinate space defined by a sensing system (such as the EM sensor system 306). To combine the sensor data and the image data on a single frame of reference (such as the graphical user interface 314), the system 300 may register the coordinate space associated with the image data (also referred to as the “image space”) with the coordinate space associated with the sensor data (also referred to as the “sensor space”). As used herein, the term “registration” refers to a mapping between different coordinate spaces. For example, the system 300 (or a registration system associated therewith) may register 332 the fluoroscopy imaging system 312 with the EM sensor system 306 by determining a mapping or spatial transformation that maps any data point in the image space to a respective data point in the sensor space. The system 300 may further use the registration 332 to dynamically update the graphical user interface 314 to reflect the current or actual spatial relationship between the instrument and the target during the intraoperative phase, as depicted by the image data captured via the fluoroscopy imaging system 312 (such as to facilitate real-time navigation).

As described above, in some medical procedures (such as bronchoscopy), the patient is under general anesthesia and allowed to breathe through a ventilator. However, because an imaging system may capture multiple images (such as tomograms) when scanning the patient's anatomy, any changes to the anatomy during the course of the scan (such as due to respiration) may introduce artifacts or other inaccuracies in the resulting images. To acquire accurate scans of a patient's anatomy during an intraoperative phase of a medical procedure, the patient may be prevented from breathing for the duration of the scan (such as by forcing a breath-hold via the ventilator). Aspects of the present disclosure recognize that respiration can change the pose (including the position and/or orientation) of a medical instrument inside the patient's anatomy. As a result, the pose of an instrument while respiration is suspended may differ from the pose of the instrument, at any given time, while the patient is breathing (even when no user inputs are applied to the instrument). Aspects of the present disclosure further recognize that some imaging technologies (such as CT or X-rays) may interfere with some sensor technologies (such as EM). Thus, sensor data acquired before scanning an anatomy may deviate from sensor data acquired after the scan. In some aspects, a medical system may compensate or otherwise account for any deviations in instrument pose caused by sensor interference or changes in anatomy when registering an image space with a sensor space and when applying the registration to real-time sensor data.

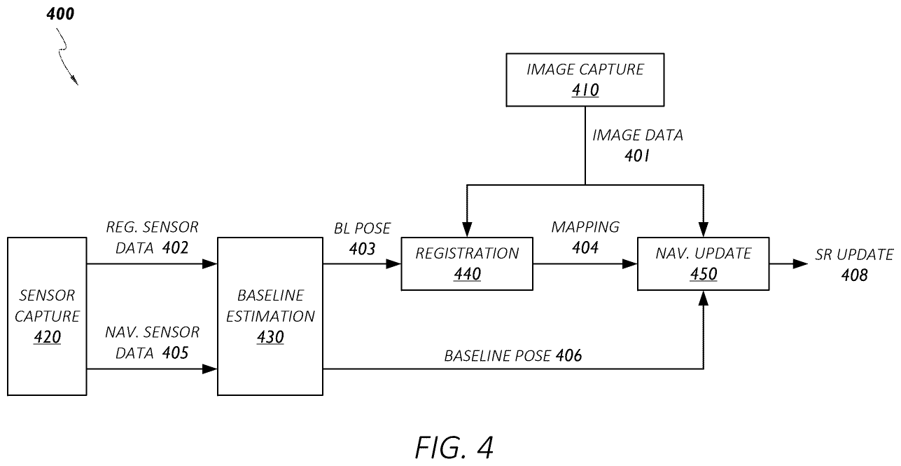

FIG. 4 shows a block diagram of an example navigation controller 400, according to some implementations. In some implementations, the navigation controller 400 may be one example of any of the control circuitry 208 or 224 of FIG. 2. The navigation controller 400 is configured to generate a spatial relationship (SR) update 408 that depicts or otherwise indicates a spatial relationship between a medical instrument and a target within an anatomy during an intraoperative phase of a medical procedure.

The navigation controller 400 includes an image capture component 410, a sensor capture component 420, a baseline estimation component 430, a registration component 440, and a navigation update component 450. The image capture component 410 is configured to interface or communicate with an imaging system (such as the fluoroscopy imaging system 312 of FIG. 3) to capture or acquire image data 401 representing one or more scans of a patient's anatomy. In some implementations, the image data 401 may include one or more tomograms captured by a CBCT scanner (such as described with reference to FIGS. 1-3). In some aspects, the image capture component 410 may capture the image data 401 while the patient is prevented from breathing. For example, the image capture component 410 may control or otherwise cause a ventilator to suspend respiration while acquiring the image data 401. In some implementations, the image capture component 410 may capture the image data 401 at the end of an inspiration phase of a respiratory cycle (such as while the anatomy is fully pressurized or expanded).

The sensor capture component 420 is configured to interface or communicate with a sensor system (such as the EM sensor system 306 of FIG. 3) to capture or acquire sensor data associated with an instrument inside the anatomy (such as an endoscope). In some implementations, the sensor data may indicate a pose of the instrument and/or one or more EM sensors disposed in an EM field (such as described with reference to FIGS. 1-3). As used herein, the term “pose” may refer to a position and/or orientation of a sensor or an instrument. Aspects of the present disclosure recognize that some imaging technologies (such as CT or X-rays) may interfere with some sensor technologies (such as EM). In some aspects, the sensor capture component 420 may capture sensor data 402 to be used for registration (also referred to as “registration sensor data”) while the imaging system cannot interfere with the underlying sensor technology (such as while the fluoroscopy imaging system 312 is located outside the EM field of the EM sensor system 306).

In other words, the registration sensor data 402 may be captured before the imaging system is brought within a proximity of the target or anatomy and before the image capture component 410 causes respiration to be suspended in the patient. Because the patient is breathing while the registration sensor data 402 is captured, the instrument pose indicated by the registration sensor data 402 may differ from the instrument pose depicted by the image data 401. More specifically, the patient's breathing may cause the instrument pose to fluctuate over time. In some aspects, the sensor capture component 420 may compensate for such discrepancies in instrument pose by capturing the registration sensor data 402 over a threshold duration so that time-varying changes in the instrument pose are reflected in the registration sensor data 402. For example, the threshold duration may be a predetermined duration configured to span at least one respiratory cycle (such as 12 seconds). In some implementations, the predetermined duration may be configured to span two or more respiratory cycles.

The baseline estimation component 430 is configured to determine a baseline (BL) pose 403 for the medical instrument based on the registration sensor data 402. In some aspects, the baseline pose 403 may represent a mean or median pose of the instrument over the threshold duration. For example, the baseline estimation component 430 may determine the mean or median pose by averaging (or determining a median of) the registration sensor data 402 accumulated over the threshold duration. However, the average pose of an instrument over one or more respiratory cycles may differ from the actual pose of the instrument at the end of an inspiration phase (such as when the image data 401 is captured). Aspects of the present disclosure recognize that the end of the inspiration phase may coincide with a peak value (or a maximum deviation) of the instrument pose over the threshold duration. Thus, in some other aspects, the baseline pose 403 may represent a peak value of the instrument pose over the threshold duration.

Aspects of the present disclosure further recognize that respiration may cause the pose of an instrument to deviate in any direction in 3D space (such as in any of the x, y, or z directions of a cartesian coordinate space). Thus, the maximum deviation of the instrument pose in any one direction may not necessarily be aligned with the end of an inspiration phase. For example, the pose of an instrument may “peak” in the x-direction before the pose of the instrument peaks in the y-direction over a given respiratory cycle. Thus, as used herein the term “peak value” specifically refers to the pose of an instrument at the end of an inspiration phase. In some implementations, the baseline estimation component 430 may determine the peak value of the instrument pose using existing signal processing techniques. Example suitable signal processing techniques may include principal component analysis (PCA), among other examples.

In some other implementations, the baseline estimation component 430 may determine the peak value of the instrument pose based at least in part on a frequency of the respiratory cycles. For example, the baseline estimation component 430 may determine a frequency response of the instrument pose by applying a Fourier transform (such as a fast Fourier transform (FFT)) to the registration sensor data 402 captured over the threshold duration. The baseline estimation component 430 may further determine the duration of a respiratory cycle based on the frequency response curve and analyze the changes in instrument pose within each respiratory cycle. For example, the baseline estimation component 430 may identify the end of an inspiration phase based on known variations or other characteristics of the instrument pose within a given respiratory cycle.

In some other implementations, the baseline estimation component 430 may determine the peak (such as a maximum or minimum) value of the instrument pose based at least in part on an average (such as a mean or median) pose of the instrument over the threshold duration. For example, aspects of the present disclosure recognize that the pose of an instrument may change much quicker during the expiration phase of a respiratory cycle (such as when the patient is breathing out) compared to the inspiration phase (such as when the patient is breathing in). As a result, the average instrument pose over a given respiratory cycle may be skewed towards the peak value associated with the inspiration phase. Thus, in some implementations, the baseline estimation component 430 may determine the peak value of the instrument pose based on a maximum deviation or displacement of the instrument from the average instrument pose.

Aspects of the present disclosure recognize that some medical systems may implement respiratory gating as a safety mechanism to prevent a medical instrument from being inserted into certain regions of a patient's airways during expiration that would likely cause trauma. More specifically, respiratory gating operations can synchronize the movement of a medical instrument with a patient's respiratory cycles. For example, one or more respiration sensors (such as EM sensors, accelerometers, and/or acoustic respiratory sensors) can be placed on a patient's body to track the respiratory cycles of the patient (including the inspiration and expiration phases of each respiratory cycle). In some implementations, the medical system may provide a visual and/or audible alert to signal the inspiration and/or expiration phases to a user, so that the user can avoid driving an instrument into an airway that is closed during an expiration phase of the respiratory cycle (where forcing the instrument into the closed airway can cause damage to the surrounding anatomy). In some implementations, the medical system may lock the robotic arms or otherwise prevent movement of the instrument into an airway that is closed during an expiration phase of the respiratory cycle. Further examples of respiratory gating are described in more detail in U.S. Pat. No. 11,490,782, titled “Robotic Systems for Navigation of Luminal Networks that Compensate for Physiological Noise,” the entirety of which is incorporated herein by reference. In some implementations, the baseline estimation component 430 may use respiratory gating to monitor the inspiration phase of the patient's respiratory cycles and determine the peak value of the instrument pose.

Aspects of the present disclosure further recognize that the peak value of the instrument pose associated with the registration sensor data 402 may still differ from the instrument pose depicted by the image data 401 due to the differences between the inspiration experienced while the patient is breathing freely compared to the inspiration experienced during a forced full-breath hold. In some implementations, the baseline estimation component 430 may use a respiratory motion model to extrapolate the instrument pose associated with a breath-hold based on the peak value of the instrument pose associated with free breathing. The respiratory motion model may be any known model that can estimate and correct for the effects of respiratory motion (such as by modeling the relationship between the motion of internal organs and the displacement of the skin surface).

In some aspects, the baseline estimation component 430 may calculate one or more confidence metrics to verify the peak value of the instrument pose across multiple spatial directions. Example suitable confidence metrics may include the root mean square (RMS) of the respiratory frequency, among other examples. For example, the baseline estimation component 430 may calculate the RMS of the respiratory frequency in the x-, y-, and z-directions to check the agreement of the computed results across all 3 directions. In some implementations, the baseline estimation component 430 may compare the confidence metric with a threshold value to determine whether the peak value is suitable for use as the baseline pose 403. In some implementations, the baseline estimation component 430 may revert to a more conservative estimate for the baseline pose 403 (such as the average instrument pose over the threshold duration) if the confidence metric is below the threshold value.

The registration component 440 is configured to receive the image data 401 and the baseline pose 403 and generate a mapping 404 between a coordinate space associated with the image data 401 (also referred to as the “image space”) and a coordinate space associated with the baseline pose 403 or the sensor data 402 (also referred to as the “sensor space”). For example, the mapping 404 may be a transformation matrix that can transform any data point (such as a coordinate or a vector) in the image space to a respective data point in the sensor space. In some implementations, the registration component 440 may determine the mapping 404 based at least in part on the baseline pose 403 of the instrument in the sensor space and a corresponding instrument pose in the image space.

In some aspects, the registration component 440 may determine the pose of the instrument in the image space through analysis of the image data 401 using one or more image processing techniques. Example suitable image processing techniques include segmentation, machine learning, and statistical analysis, among other examples. As used herein, the term “segmentation” refers to various techniques for partitioning a digital image into groups of voxels (or “image segments”) based on related characteristics or identifying features. In some implementations, the registration component 440 may segment the image data 401 so that the pose of the instrument can be detected or estimated from the corresponding images (or tomograms).

The navigation update component 450 is configured to determine the updated spatial relationship 408 between the instrument and a target (such as a nodule) within the anatomy based at least in part on the image data 401 and the mapping 404. In some aspects, the navigation update component 450 may determine a position of the target in the image space through segmentation of the image data 401 (such described above). The navigation update component 450 may determine a pose of the instrument in the sensor space based on real-time navigation sensor data 405 captured by the sensor capture component 420. The navigation sensor data 405 includes any sensor data that is received via the sensor system and used for navigating the instrument within the anatomy (including sensor data received while the fluoroscopy imaging system 312 is located in or proximate to the EM field of the EM sensor system 306). The navigation update component 450 may use the mapping 404 to transform the position of the target in the image space to a corresponding position in the sensor space (or to transform the instrument pose in the sensor space to a corresponding pose in the image space) to determine the real-time spatial relationship 408 between the target and the instrument.

As described above, some imaging technologies (such as CT or X-rays) may interfere with some sensor technologies (such as EM). As a result, the navigation sensor data 405 may further deviate (compared to the registration sensor data 402) due to interference caused by the imaging system or changes to the anatomy. Thus, in some implementations, the sensor capture component 420 may capture the navigation sensor data 405 over a threshold duration (which may be the same threshold duration for capturing the registration sensor data 402) to accumulate any changes or variations in instrument pose over one or more respiratory cycles so that the baseline estimation component 430 can determine a new baseline pose 406 for the navigation sensor data 405. In some implementations, the new baseline pose 406 may represent an average instrument pose over the threshold duration. In some other implementations, the new baseline pose 406 may represent a peak value of the instrument pose over the threshold duration.

The navigation update component 450 may apply the real-time spatial relationship 408 to the new baseline pose 406 to adjust the pose of the instrument or adjust the location of the target in a graphical interface (such as the graphical user interface 314 of FIG. 3). As a result, the updated graphical interface may depict a more accurate spatial relationship between the instrument and the target while accounting for any deviations in instrument pose caused by sensor interference or changes in anatomy when registering the image space with the sensor space or when applying the registration to real-time sensor data.

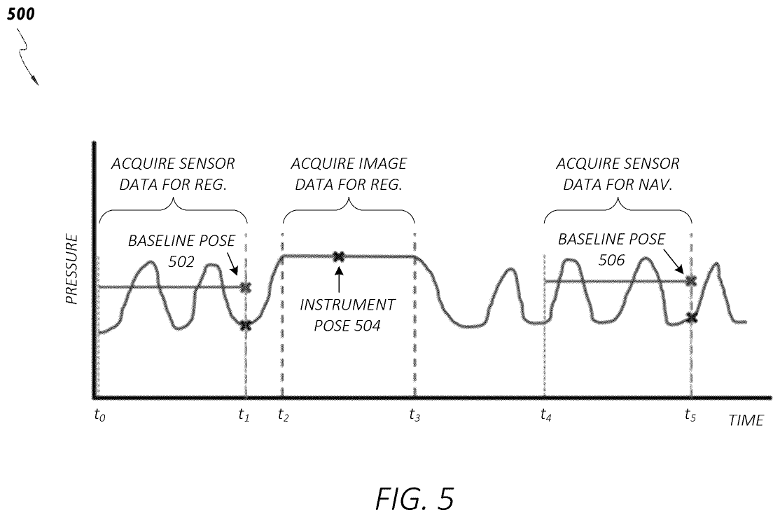

FIG. 5 shows an example timing diagram 500 depicting changes in pressure inside a patient anatomy during a medical procedure. For example, the changes in pressure may be caused by a patient's respiration. As described with reference to FIG. 4, such changes in pressure may be correlated with changes to the pose of an instrument inside the anatomy. In some implementations, a navigation controller (such as the navigation controller 400 of FIG. 4) may compensate for such changes or differences in instrument pose when registering an image space with a sensor space and when applying the registration to real-time sensor data.

With reference for example to FIG. 4, the sensor capture component 420 may capture registration sensor data 402 over a threshold duration, between times t₀ and t₁, while the patient is freely breathing and the imaging system is located beyond a proximity of the anatomy (so as not to interfere with the sensor data). In the example of FIG. 5, the threshold duration is shown to span 2 respiratory cycles. At time t₁, the baseline estimation component 430 may determine a first baseline pose 502 for the instrument based on the sensor data 402 accumulated over the threshold duration. In some implementations, the first baseline pose 502 may be one example of the baseline pose 403 of FIG. 4. In the example of FIG. 5, the first baseline pose 502 is depicted as an average pose of the instrument over the threshold duration. However, in some other implementations, the first baseline pose 502 may be a peak value of the instrument pose over the threshold duration (such as described with reference to FIG. 4).

Between times t₁ and t₂, the imaging system may be brought into the proximity of the anatomy. The image capture component 410 may capture image data 401 (via the imaging system), between times t₂ and t₃, while suspending respiration in the patient (such as by forcing a breath-hold via a ventilator). As a result, the instrument pose 504 in the image space may deviate from the instrument pose in the sensor space at any given time over the threshold duration. However, compared to the instrument pose detected at the end of an expiration phase of a respiratory cycle (such as at time t₁), the baseline pose 502 is substantially closer to the instrument pose 504 in the image space. As described with reference to FIG. 4, the baseline estimation component 430 may implement various other compensation techniques (such as peak value detection or respiratory modeling) to further close the gap between the baseline pose 502 and the instrument pose 504 in the image space.

Between times t₃ and t₄, the registration component 440 may determine a mapping 404 between the image space and the sensor space based at least in part on the baseline pose 502 and the instrument pose 504 in the image space. During this time, the patient is allowed to breathe freely again, however, the proximity of the imaging system to the patient anatomy may interfere with the any sensor data captured after time t₃. Thus, the sensor capture component 420 may capture navigation sensor data 405 over a threshold duration, between times t₄ and t₅, to compensate for any subsequent deviations in instrument pose. At time t₅, the baseline estimation component 430 may determine a second baseline pose 506 for the instrument based on the sensor data 405 accumulated over the threshold duration. In some implementations, the second baseline pose 506 may be one example of the baseline pose 406 of FIG. 4. In the example of FIG. 5, the second baseline pose 506 is depicted as an average pose of the instrument over the threshold duration. However, in some other implementations, the second baseline pose 506 may be a peak value of the instrument pose over the threshold duration (such as described with reference to FIG. 4).

At time t₅, the navigation update component 450 may apply the mapping 404 to the second baseline pose 506 to determine a spatial relationship 408 between the instrument and a target within the anatomy. For example, the navigation update component 450 may determine a position of the target in the image space through segmentation of the image data 401. The navigation update component 450 may use the mapping 404 to transform the position of the target in the image space to a corresponding position in the sensor space (or to transform the second baseline pose 506 in the sensor space to a corresponding pose in the image space) to determine the spatial relationship 408 between the instrument and the target (such as described with reference to FIG. 4).

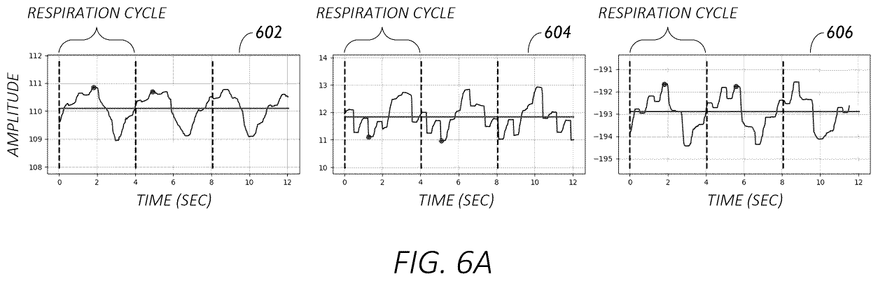

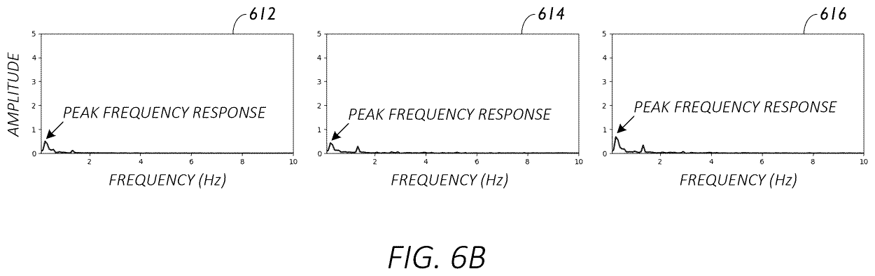

FIG. 6A shows example timing diagrams 602-606 depicting the pose of an instrument in response to changes in pressure. More specifically, the timing diagrams 602-606 show changes in the instrument pose over a 12-second duration of time (T) along the x-, y-, and z-directions, respectively, of a cartesian coordinate space. FIG. 6B shows example frequency diagrams 612-616 depicting a frequency response of the time-varying poses of the instrument shown in FIG. 6A. For example, the frequency diagrams 612-616 may be generated by a applying a Fourier transform (such as an FFT) to the timing diagrams 602-606, respectively, of FIG. 6A.

With reference for example to FIG. 6B, each of the frequency response curves along the x-, y-, and z-axes peaks at ˜0.25 Hz. In other words, each 1-second interval of the timing diagrams 602-606 depicts ¼ of a complete respiratory cycle. With reference for example to FIG. 6A, the 12-second duration in each of the timing diagrams 602-606 can be subdivided into three 4-second intervals each corresponding to a full respiration cycle. Aspects of the present disclosure recognize that, within a respiration cycle, the inspiration phase is generally associated with a more gradual change in instrument pose (such as shown between times T=0 and T=2) whereas the expiration phase is generally associated with a more sudden change in instrument pose (such as shown between times T=2 and T=3). Based on these characteristics, a baseline estimation system (such as the baseline estimation component 430 of FIG. 4) can estimate the peak value of the instrument pose that coincides with the end of the respiration phase. In the example of FIG. 6A, the peak value is shown to occur around 2 seconds and 5 seconds (depicted by dots in the timing diagrams 602-606).

Additionally, the timing diagrams 602-606 of FIG. 6A show the average instrument pose in each of the x-, y-, and z-directions (depicted as a horizontal line having an amplitude of approximately 110, 12, and −193 in the timing diagrams 602-606, respectively). As described above, the inspiration phase of a respiration cycle is generally associated with a more gradual change in instrument pose than the expiration phase, which causes the average instrument pose to be skewed towards the instrument poses detected during the inspiration phase. For example, as shown in FIG. 6A, the average instrument pose in the x-direction is closer to the highest point of the respiration curve compared to the lowest point (as shown in the timing diagram 602), whereas the average instrument pose in the y-direction is closer to the lowest point of the respiration curve compared to the highest point (as shown in the timing diagram 604). The baseline estimation system also may use these characteristics to estimate (or verify) the peak value of the instrument pose that coincides with the end of the inspiration phase.

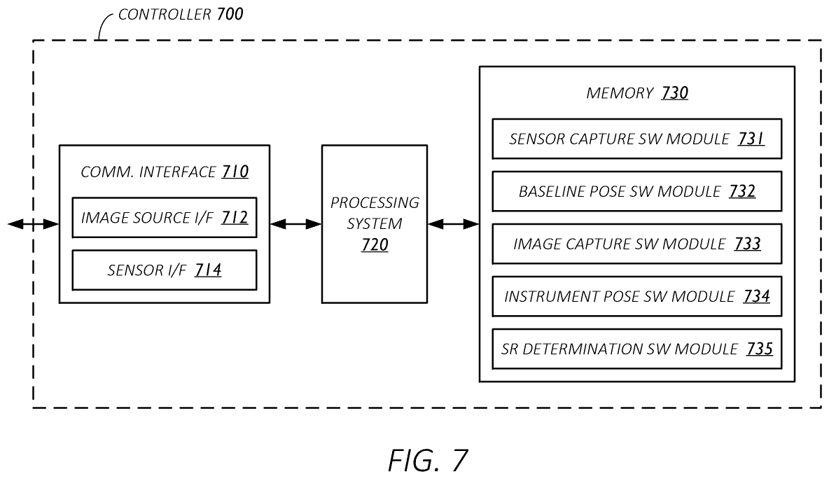

FIG. 7 shows a block diagram of an example controller 700 for a medical system, according to some implementations. In some implementations, the controller 700 may be one example of the navigation controller 400 of FIG. 4. More specifically, the controller 700 is configured to determine a spatial relationship between an instrument and a target within an anatomy of a patient during an intraoperative phase of a medical procedure.

The controller 700 includes a communication interface 710, a processing system 720, and a memory 730. The communication interface 710 is configured to communicate with one or more components of the medical system. More specifically, the communication interface 710 includes an image source interface (I/F) 712 for communicating with one or more image sources (such as the CT imaging system 310 and/or the fluoroscopy imaging system 312 of FIG. 3) and a sensor interface (I/F) 714 for communicating with one or more sensors (such as the EM sensor system 306 of FIG. 3).

The memory 730 may include a non-transitory computer-readable medium (including one or more nonvolatile memory elements, such as EPROM, EEPROM, Flash memory, or a hard drive, among other examples) that may store the following software (SW) modules: a sensor capture SW module 731 to capture sensor data, over a threshold duration spanning one or more cyclic movements of an object (such as an anatomy), via a sensor disposed on the instrument; a baseline pose SW module 732 to determine a baseline pose of the instrument in a first coordinate space based on the sensor data captured over the threshold duration; an image capture SW module 733 to capture image data via an imaging system external to the object while movement is suspended following the threshold duration; an instrument pose SW module 734 to determine a pose of the instrument in a second coordinate space based on the image data captured after the threshold duration; and a spatial relationship (SR) determination SW module 735 to determine a spatial relationship between the instrument and a target within the object based at least in part on the baseline pose of the instrument in the first coordinate space and the pose of the instrument in the second coordinate space. Each of the software modules 731-735 includes instructions that, when executed by the processing system 720, causes the controller 700 to perform the corresponding functions.

The processing system 720 may include any suitable one or more processors capable of executing scripts or instructions of one or more software programs stored in the controller 700 (such as in the memory 730). For example, the processing system 720 may execute the sensor capture SW module 731 to capture sensor data, over a threshold duration spanning one or more cyclic movements of an object, via a sensor disposed on the instrument. The processing system 720 also may execute the baseline pose SW module 732 to determine a baseline pose of the instrument in a first coordinate space based on the sensor data captured over the threshold duration. The processing system 720 may execute the image capture SW module 733 to capture image data via an imaging system external to the object while movement is suspended following the threshold duration. The processing system 720 also may execute the instrument pose SW module 734 to determine a pose of the instrument in a second coordinate space based on the image data captured after the threshold duration. The processing system 720 may further execute the SR determination SW module 735 to determine a spatial relationship between the instrument and a target within the object based at least in part on the baseline pose of the instrument in the first coordinate space and the pose of the instrument in the second coordinate space.

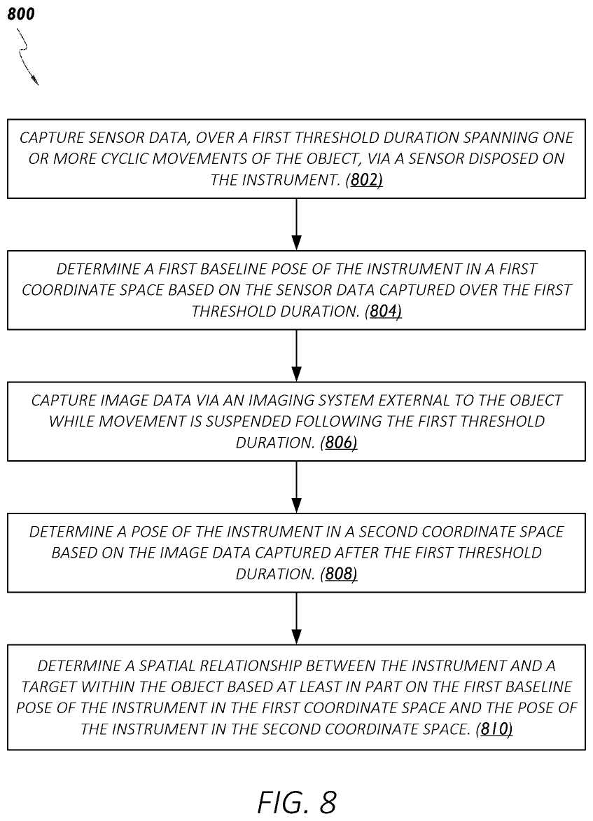

FIG. 8 shows an illustrative flowchart depicting an example operation 800 for navigating an instrument within an object, according to some implementations. In some implementations, the example operation 800 may be performed by a controller for a medical system such as the controller 700 of FIG. 7 or the navigation controller 400 of FIG. 4.

The controller may capture sensor data, over a first threshold duration spanning one or more cyclic movements of the object, via a sensor disposed on the instrument (802). The controller may determine a first baseline pose of the instrument in a first coordinate space based on the sensor data captured over the first threshold duration (804). The controller may capture image data via an imaging system external to the object while movement is suspended following the first threshold duration (806). In some implementations, the suspension of movement may coincide with the end of an inspiration phase of a respiratory cycle following the first threshold duration. The controller may determine a pose of the instrument in a second coordinate space based on the image data captured after the first threshold duration (808). The controller may further determine a spatial relationship between the instrument and a target within the object based at least in part on the first baseline pose of the instrument in the first coordinate space and the pose of the instrument in the second coordinate space (810).

In some aspects, the first baseline pose may be determined based at least in part on a respiratory model and the sensor data captured over the first threshold duration. In some implementations, the controller may further determine a maximum deviation of the sensor over the first threshold duration based at least in part on an average of the sensor data and may select a subset of the sensor data that coincides with the maximum deviation of the sensor, where the first baseline pose is determined based on the selected subset of sensor data.

In some aspects, the one or more cyclic movements may be associated with one or more respiratory cycles. In some implementations, the controller may further determine one or more inspiration phases of the one or more respiratory cycles, respectively, based at least in part on the sensor data and may select a respective subset of the sensor data that coincides with the end of each of the one or more inspiration phases, where the first baseline pose is determined based on the selected subsets of sensor data. In some implementations, the controller may further determine a frequency of the one or more respiratory cycles based on the sensor data, where the one or more inspiration phases is determined based on the frequency of the one or more respiratory cycles. In some implementations, the one or more inspiration phases may be determined based on a respiratory gating operation that synchronizes a movement of the instrument with the one or more respiratory cycles.

In some aspects, the determining of the spatial relationship between the instrument and the target may include determining a mapping between the first coordinate space and the second coordinate space based at least in part on the first baseline pose of the instrument in the first coordinate space and the pose of the instrument in the second coordinate space. In some implementations, the determining of the spatial relationship between the instrument and the target may further include capturing sensor data via the sensor over a second threshold duration following the capture of the image data, determining a second baseline pose of the instrument in the first coordinate space based on the sensor data captured over the second threshold duration, and applying the mapping to the second baseline pose. In some implementations, the second threshold duration may span one or more respiratory cycles.

Those of skill in the art will appreciate that information and signals may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative logics, logical blocks, modules, circuits and algorithm processes described in connection with the implementations disclosed herein may be implemented as electronic hardware, computer software, or combinations of both. The interchangeability of hardware and software has been described generally, in terms of functionality, and illustrated in the various illustrative components, blocks, modules, circuits and processes described herein. Whether such functionality is implemented in hardware or software depends upon the particular application and design constraints imposed on the overall system.

In the foregoing specification, implementations have been described with reference to specific examples thereof. It will, however, be evident that various modifications and changes may be made thereto without departing from the broader scope of the disclosure as set forth in the appended claims. The specification and drawings are, accordingly, to be regarded in an illustrative sense rather than a restrictive sense.

As used herein, a phrase referring to “at least one of” a list of items refers to any combination of those items, including single members. As an example, “at least one of: a, b, or c” is intended to cover: a, b, c, a-b, a-c, b-c, and a-b-c.

Various modifications to the implementations described in this disclosure may be readily apparent to those skilled in the art, and the generic principles defined herein may be applied to other implementations without departing from the spirit or scope of this disclosure. Thus, the claims are not intended to be limited to the implementations shown herein, but are to be accorded the widest scope consistent with this disclosure, the principles and the novel features disclosed herein.

What is claimed is:

1. A method for navigating an instrument within an object, comprising:
capturing sensor data, over a first threshold duration spanning one or more cyclic movements of the object, via a sensor disposed on the instrument;
determining a first baseline pose of the instrument in a first coordinate space based on the sensor data captured over the first threshold duration;
capturing image data via an imaging system external to the object while movement is suspended following the first threshold duration;
determining a pose of the instrument in a second coordinate space based on the image data captured after the first threshold duration; and
determining a spatial relationship between the instrument and a target within the object based at least in part on the first baseline pose of the instrument in the first coordinate space and the pose of the instrument in the second coordinate space.

2. The method of claim 1, wherein the first baseline pose is determined based at least in part on a respiratory model and the sensor data captured over the first threshold duration.

3. The method of claim 2, further comprising:
determining a maximum deviation of the sensor over the first threshold duration based at least in part on an average of the sensor data; and
selecting a subset of the sensor data that coincides with the maximum deviation of the sensor, the first baseline pose being determined based on the selected subset of sensor data.

4. The method of claim 1, wherein the suspension of movement coincides with the end of an inspiration phase of a respiratory cycle following the first threshold duration.

5. The method of claim 1, wherein the one or more cyclic movements are associated with one or more respiratory cycles.

6. The method of claim 5, further comprising:
determining one or more inspiration phases of the one or more respiratory cycles, respectively, based at least in part on the sensor data; and
selecting a respective subset of the sensor data that coincides with the end of each of the one or more inspiration phases, the first baseline pose being determined based on the selected subsets of sensor data.

7. The method of claim 6, further comprising:
determining a frequency of the one or more respiratory cycles based on the sensor data, the one or more inspiration phases being determined based on the frequency of the one or more respiratory cycles.

8. The method of claim 6, wherein the one or more inspiration phases are determined based on a respiratory gating operation that synchronizes a movement of the instrument with the one or more respiratory cycles.

9. The method of claim 1, wherein the determining of the spatial relationship between the instrument and the target comprises:
determining a mapping between the first coordinate space and the second coordinate space based at least in part on the first baseline pose of the instrument in the first coordinate space and the pose of the instrument in the second coordinate space.

10. The method of claim 9, wherein the determining of the spatial relationship between the instrument and the target further comprises:
capturing sensor data via the sensor over a second threshold duration following the capture of the image data;
determining a second baseline pose of the instrument in the first coordinate space based on the sensor data captured over the second threshold duration; and
applying the mapping to the second baseline pose.

11. The method of claim 10, wherein the second threshold duration spans one or more respiratory cycles.

12. A controller for a medical system, comprising:
a processing system; and
a memory storing instructions that, when executed by the processing system, cause the controller to:
capture sensor data, over a first threshold duration spanning one or more cyclic movements of an object, via a sensor disposed on the instrument;
determine a first baseline pose of the instrument in a first coordinate space based on the sensor data captured over the first threshold duration;
capture image data via an imaging system external to the object while movement is suspended following the first threshold duration;
determine a pose of the instrument in a second coordinate space based on the image data captured after the first threshold duration; and
determine a spatial relationship between the instrument and a target within the object based at least in part on the first baseline pose of the instrument in the first coordinate space and the pose of the instrument in the second coordinate space.

13. The controller of claim 12, wherein the first baseline pose is determined based at least in part on a respiratory model and the sensor data captured over the first threshold duration.

14. The controller of claim 13, wherein execution of the instructions further causes the controller to:
determine a maximum deviation of the sensor over the first threshold duration based at least in part on an average of the sensor data; and
select a subset of the sensor data that coincides with the maximum deviation of the sensor, the first baseline pose being determined based on the selected subset of sensor data.

15. The controller of claim 12, wherein the suspension of movement coincides with the end of an inspiration phase of a respiratory cycle following the first threshold duration.

16. The controller of claim 12, wherein the one or more cyclic movements are associated with one or more respiratory cycles.

17. The controller of claim 16, wherein execution of the instructions further causes the controller to:
determine one or more inspiration phases of the one or more respiratory cycles, respectively, based at least in part on the sensor data; and
select a respective subset of the sensor data that coincides with the end of each of the one or more inspiration phases, the first baseline pose being determined based on the selected subsets of sensor data.

18. The controller of claim 17, wherein execution of the instructions further causes the controller to:
determine a frequency of the one or more respiratory cycles based on the sensor data, the one or more inspiration phases being determined based on the frequency of the one or more respiratory cycles.

19. The controller of claim 12, wherein the determining of the spatial relationship between the instrument and the target comprises:
determining a mapping between the first coordinate space and the second coordinate space based at least in part on the first baseline pose of the instrument in the first coordinate space and the pose of the instrument in the second coordinate space.

20. The controller of claim 19, wherein the determining of the spatial relationship between the instrument and the target further comprises:
capturing sensor data via the sensor over a second threshold duration following the capture of the image data;
determining a second baseline pose of the instrument in the first coordinate space based on the sensor data captured over the second threshold duration; and
applying the mapping to the second baseline pose.
